(19) United States
(12) Patent Application Publication
(10) Pub. No.: US 2018/0353484 A1
(43) Pub. Date: Dec. 13, 2018

(54) USAGE OF ACETAZOLAMIDE FOR TREATING ICH, PARTICULARLY HYPERGLYCEMIC ICH

(71) Applicant: CHENG-DI CHIU, Taichung City (TW)

(72) Inventor: CHENG-DI CHIU, Taichung City (TW)

(21) Appl. No.: 15/621,489

(22) Filed: Jun. 13, 2017

(51) Int. Cl. A61K 31/433

(52) U.S. Cl. CPC A61K 31/433

(57) ABSTRACT

A use of acetazolamide for treatment of hyperglycemic intracerebral hemorrhage (ICH). The acetazolamide is able to reduce the hemorrhagic area of intracerebral hemorrhage, so as to efficiently treat intracerebral hemorrhage.

### BACKGROUND OF THE INVENTION

### 1. Field of the Invention

The present invention relates to use of compounds, and more particularly, to a use of acetazolamide for treating intracerebral hemorrhage (ICH).

### 2. Description of the Related Art

Intracerebral hemorrhage (ICH) remains a devastating disease with significant mortality rate. Patients with diabetes mellitus or hyperglycemia who experience ICH have been shown to have increased mortality and poorer neurological outcomes. Detrimental effects of hyperglycemia in experimental ICH have been also reported. Hyperglycemia in experimental ICH induced increased vasogenic brain edema and worsened neurobehavioral outcomes. High blood glucose concentration increased brain edema and level of apoptosis in perihematomal regions after ICH. As a result, the recovering status of the hemorrhagic area is adversely affected.

Acetazolamide (AZA) is a medicine for lowering intraocular pressure, treating glaucoma, and relieving headache caused by high altitude disease, stomach depression, and dyspnea. Also, acetazolamide is used to assist the treatment of epilepsy and edema.

Further, acetazolamide is a carbonic anhydrase inhibitor, with an operation mechanism of enhancing urine flow and excretion of bicarbonate. Acetazolamide has been proven to inhibit an activity of Aquaporin-4 (AQP4) as well as reduce brain edema and neuronal death after an intracerebral hemorrhage. However, the roles of AZA in hyperglycemic ICH have not been addressed.

Therefore, by exploiting the usage of acetazolamide for treating ICH, the application scope of acetazolamide is advantageously improved.

### SUMMARY OF THE INVENTION

For improving the issues above, a use of acetazolamide for treatment of hyperglycemic intracerebral hemorrhage (ICH) is provided. By treating the patient of hyperglycemic ICH with acetazolamide, hemorrhagic area is reduced or recovered.

An embodiment of the present invention provides a usage of acetazolamide compound for treating ICH, which is used for preparing the medicine for treating ICH.

An embodiment of the present invention provides a usage of acetazolamide compound for treating ICH, which is used for preparing the medicine for treating hyperglycemic ICH.

Preferably, the acetazolamide compound is able to inhibit the activity of AQP4.

Preferably, the acetazolamide compound is capable of reducing the hemorrhagic area.

Preferably, the acetazolamide compound is administered by oral administration or injection.

Detailed description of technical features and embodiments are provided in the following paragraphs. People with ordinary skills in the field of the present invention are allowed to understand the technical features accordingly.

### BRIEF DESCRIPTION OF THE DRAWINGS

FIG. 1 is a schematic diagram illustrating the experimental result of the plasma glucose levels in normoglycemic and hyperglycemic rats with intracerebral hemorrhage treated with or without AZA.

FIG. 2A is a schematic diagram illustrating the gross hemorrhagic area from brains of normoglycemic and hyperglycemic rats with experimental ICH treated with or without AZA.

FIG. 2B is a diagram illustrating the percentage of calculated hemorrhagic area of brains of normoglycemic and hyperglycemic rats with experimental ICH treated with or without AZA.

FIG. 3 is a diagram illustrating the experimental result of the expression of AQP4 in the brain tissues of normoglycemic and hyperglycemic rats with experimental ICH treated with AZA.

### DETAILED DESCRIPTION OF THE INVENTION

The aforementioned and further advantages and features of the present invention will be understood by reference to the description of the preferred embodiment in conjunction with the accompanying drawings.

The compound herein disclosed shall be understood as possessing specific functions, and the specific structure herein disclosed is required for performing such functions. It shall be understood that various structures related to the structure herein disclosed are able to perform identical or similar function, so as to acquire identical or similar effects.

A usage of acetazolamide (AZA) for treating ICH is provided by the present invention. In an embodiment of the present invention, the AZA compound is applied for preparing a medicine which used for treating patients having ICH. In another embodiment, the AZA compound is applied for preparing a medicine used for specifically treating patients having hyperglycemic ICH.

Embodiments and relative experiments provide a comprehensive disclosure and description regarding the compound, the prepared composition, and the method of preparation and assessment in accordance with the claims of the present invention. Such disclosures shall not limit the content of embodiments and scope of the claims.

Experiment 1: showing plasma glucose levels in normoglycemic and hyperglycemic rats with intracerebral hemorrhage treated with or without AZA.

In the hyperglycemia experiment, six Wistar rats are applied. Regarding each rat, hyperglycemia is induced by a single intraperitoneal injection of a buffer solution of streptozotocin (STZ) in 31 mM citrate and 39 mM sodium phosphate at 70 mg/kg body weight after an overnight fast. On the other hand, control rats receive an intraperitoneal injection of buffer vehicle. On day 3 after STZ treatment, whole blood of the rats are obtained and the glucose levels thereof is measured. Blood glucose level of ≥250 mg/dL was chosen as hyperglycemia, whereas rats were used as normoglycemia, defined as a blood glucose level of <100 mg/dL.

Then in the intracerebral hemorrhage experiment, each hyperglycemic or normoglycemic rat is anesthetized with an intraperitoneal injection of 50 mg/kg pentobarbital. Next, a needle is introduced through a burr hole into the striatum, and the needle is then removed, with the burr hole sealed with bone wax and the wound sutured. After a period of time, AZA is administered orally or by gastric lavage at 5 mg/kg in normal saline. Blood of the rats are sampled on days 1, 4, and 7 post-induction of ICH for testing the glucose level thereof.

TABLE 1
glucose levels of the rats

| | days | | | |
|---|---|---|---|---|
| category | 0 | 1 | 4 | 7 |
| normoglycemic | 80.75 ± | 117.75 ± | 132.25 ± | 117.25 ± |
| rats treated | 4.03 mg/dL | 7.59 mg/dL | 17.06 mg/dL | 9.00 mg/dL |
| with AZA | | | | |
| hyperglycemic | 298.75 ± | 386.75 ± | 313.00 ± | 381.25 ± |
| rats treated | 27.85 mg/dL | 38.89 mg/dL | 70.86 mg/dL | 76.93 mg/dL |
| with AZA | | | | |

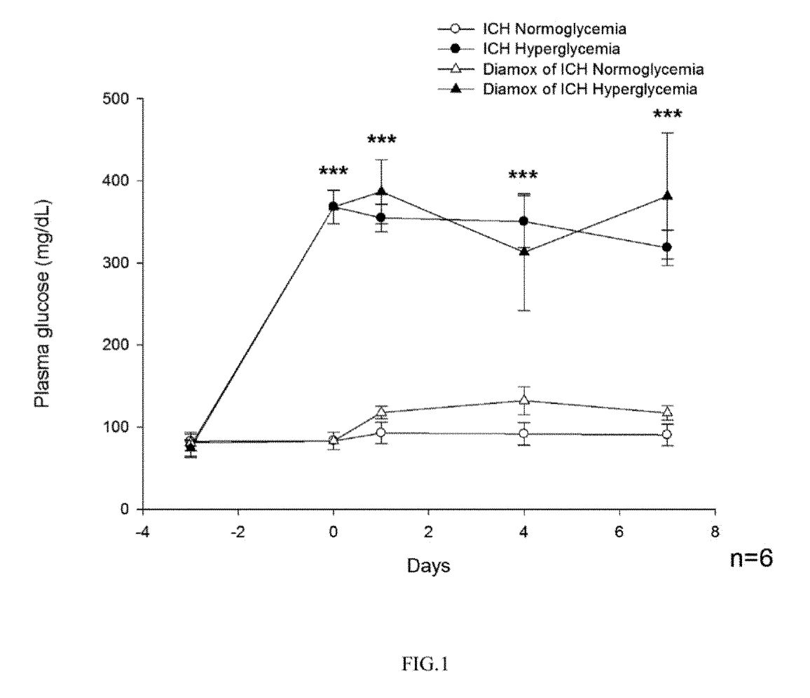

As shown by Table 1 and FIG. 1 when the AZA is administered, both the blood glucose levels of normoglycemic and hyperglycemic rats slightly rise, but do not deteriorate the intracerebral hemorrhage situation.

Experiment 2: comparing the hemorrhagic areas of normoglycemic and hyperglycemic rats treated with AZA.

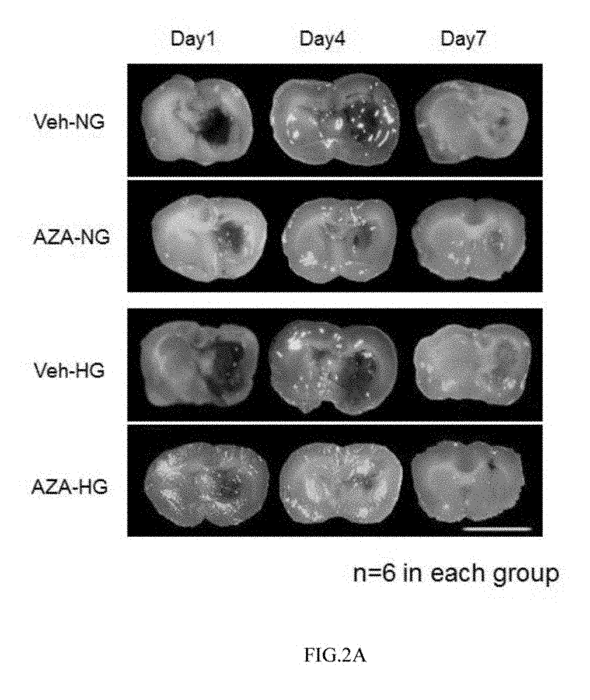

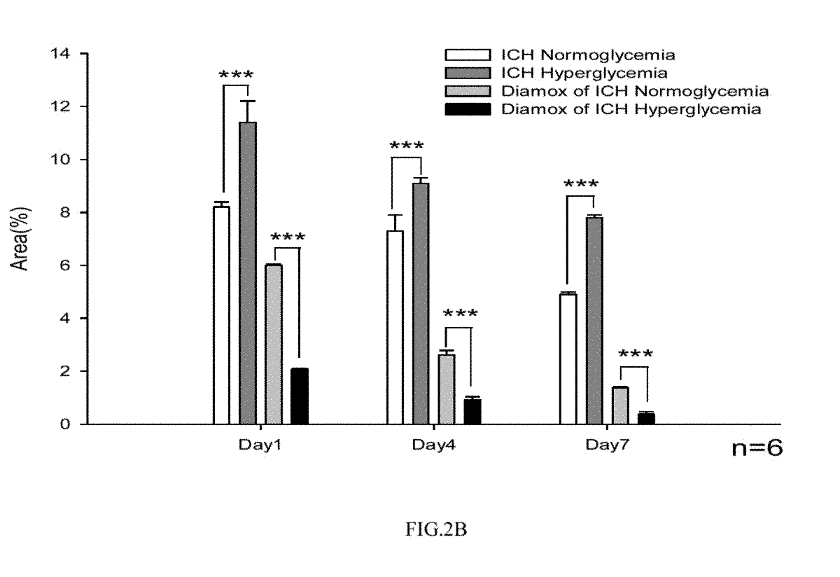

Referring to FIG. 2A, after a long observation, the hemorrhagic areas of normoglycemic and hyperglycemic rats treated with AZA become smaller, and the measurement statistics of hemorrhagic areas are listed in Table 2.

TABLE 2
hemorrhagic areas square measure of rats (%)

| | days | | |
|---|---|---|---|
| category | 1 | 4 | 7 |
| normoglycemic | 6.00 ± 0.05% | 2.61 ± 0.17% | 1.37 ± 0.05% |
| rats treated | | | |
| with AZA | | | |
| hyperglycemic | 2.08 ± 0.02% | 0.91 ± 0.13% | 0.38 ± 0.10% |
| rats treated | | | |
| with AZA | | | |

As shown by Table 2 and FIG. 2B, both the hemorrhagic areas of normoglycemic and hyperglycemic rats show a significant decrease; beside, AZA provides a significant and efficient treating effect against hemorrhage in hyperglycemic rats.

Experiment 3: determining the expression of AQP4 in normoglycemic and hyperglycemic rats.

The differential expressions of AQP4 in normoglycemic and hyperglycemic rats are detected by AQP4 autoantibody by Western blotting. The effect of AZA influencing the activity of AQP4 are tested.

TABLE 3
the expression of AQP4 in normoglycemic and hyperglycemic rats

| | days | | |
|---|---|---|---|
| category | 1 | 4 | 7 |
| normoglycemic | 12.19 ± 2.75 | 18.56 ± 5.67 | 37.84 ± 6.53 |
| rats treated | | | |
| with AZA | | | |
| hyperglycemic | 15.67 ± 6.59 | 8.65 ± 4.53% | 2.33 ± 3.83 |
| rats treated | | | |
| with AZA | | | |

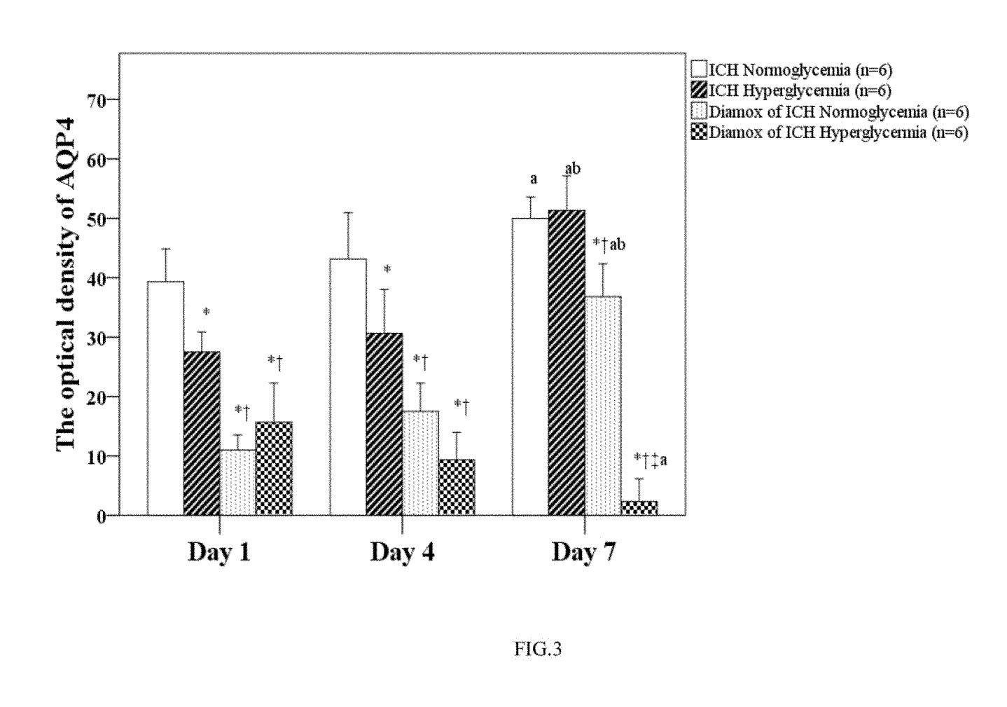

As shown by Table 3 and FIG. 3, when hyperglycemic rats is administered with AZA (5 mg/kg), the AQP4 expression shows a significant decrease. Therefore, AZA is able to directly or indirectly inhibit the activity of AQP4, thus providing a sufficient and effective treating efficacy against ICH.

Although particular embodiments of the invention have been described in detail for purposes of illustration, various modifications and enhancements may be made without departing from the spirit and scope of the invention. Accordingly, the invention is not to be limited except as by the appended claims.

1-8. (canceled)

9. A method of treating hyperglycemic intracerebral hemorrhage (ICH) with acetazolamide, wherein an amount of 5 mg/kg of acetazolamide is administered by oral or injection manner, so as to reduce a hemorrhagic area of a hyperglycemic intracerebral hemorrhage patient.

10. The method of claim 9, wherein the acetazolamide inhibits an activity of Aquaporin-4 (AQP4).
